(19) United States
(12) Patent Application Publication
(10) Pub. No.: US 2008/0086162 A1
(43) Pub. Date: Apr. 10, 2008

(54) SOFT TISSUE DISSECTOR

(75) Inventors: Gary Douglas, Billerica, MA (US); David Regan, Pelham, NH (US); Nilendu Srivastava, Chelmsford, MA (US); Rachana Suchdev, Nashua, NH (US)

Correspondence Address: Vern Maine & Associates, 100 MAIN STREET, P O BOX 3445, NASHUA, NH, 03061-3445, US

(73) Assignee: VASCULAR TECHNOLOGY INC., Nashua, NH (US)

(21) Appl. No.: 11/869,351

(22) Filed: Oct. 9, 2007

Related U.S. Application Data
(60) Provisional application No. 60/828,480, filed on Oct. 6, 2006.

(51) Int. Cl. A61B 17/00

(52) U.S. Cl. 606/190

(57) ABSTRACT

A system for soft tissue dissection, the system comprising: an elongate handle and a synthetic, non-absorbent, tip disposed on said handle.

### RELATED APPLICATIONS

This application claims the benefit of U.S. Provisional Applications No. 60/828,480, filed Oct. 6, 2006. This application is herein incorporated in its entirety by reference.

### FIELD OF THE INVENTION

The invention relates to soft tissue dissectors, and more particularly, to a soft tissue dissector configured for use in laparoscopy.

### BACKGROUND OF THE INVENTION

Soft tissue dissectors also known as Kittner dissectors, have been employed in open surgery at least since the Civil War. Such dissectors perform the double function of absorbing blood to improve visibility in the incision and to develop dissection planes between tissues. Other means used to induce such planes include digital or manual separation by the surgeon, a much cruder and often times less successful technique of blunt dissection.

Minimally invasive surgical techniques, such as laparoscopy and endoscopy have become increasingly prevalent as the benefits of decreased patient morbidity and recovery time have been recognized. These techniques, however, decrease access to the surgical site, requiring the surgeon to substitute direct digital contact with tissue with thin-shafted laparoscopic probes or instruments that are over 4 inches in length. Likewise deteriorated, saturated, or rarely, lost dissectors must be replaced through small incisions, wasting time, increasing expense, and risking added morbidity. In addition, the Kittner as designed for open surgery, assumes dissection in an often blood-tinged field. Cotton gauze or spun cotton represents a highly absorbent material that has traditionally been employed for soft tissue dissectors in open surgical procedures. Their absorbency allows the surgeon to clear away blood in the typically blood tinged fields that are unavoidable in open surgery, and thus makes them well suited for these procedures. Typically, cotton is tightly spun into a dense ball that provides it with adequate rigidity to function as an efficient soft tissue dissector. However, with saturation, cotton based dissecting tips soften and rapidly lose their rigid structure; this leads to a deterioration of dissecting function. In contrast, with minimally invasive laparoscopic surgery, the pressure induced by the pneumoperitoneum, in addition to other clinical factors unique to minimally invasive surgery, largely precludes venous oozing and bleeding, thereby decreasing the need to clear the field of blood. Hence the traditional absorbency of the Kittner, which results in saturation and deterioration of functionality, is less necessary in laparoscopy.

Furthermore, on occasion, the dissecting tip of currently used soft tissue dissectors detaches from the handle and falls into the surgical field, requiring retrieval. Removal of the soft tissue dissector from the laparoscopic port presents the greatest risk of detachment of the dissecting tip—if the dissector is not properly centered within the port channel during removal, the edge or wall of the port can exert excessive mechanical force on the dissecting tip. The likelihood of such an occurrence is also greatly increased when a dissecting tip becomes saturated, causing loss of structural strength. While retrieval of a relatively small component such as a dissecting tip may not be problematic in open surgical procedures, in laparoscopic procedures, the retrieval of such a component can be astonishingly time consuming and problematic. In fact, this complication can not only lead to increased procedure time and frustration, but also increased patient morbidity.

What is needed, therefore, are techniques for soft tissue dissection adapted for use in laparoscopic and endoscopic techniques. These dissectors would be light-weight, non-absorbent, and durable.

### SUMMARY OF THE INVENTION

One embodiment of the invention provides a system for soft tissue dissection, the system comprising: an elongated handle and a synthetic, minimally absorbent or substantially non-absorbent, tip disposed on said handle.

Another embodiment of the present invention provides such a system wherein said tip is sufficiently durable to resist deterioration throughout a surgical procedure.

Even another embodiment of the present invention provides such a system wherein said tip is of minimally or substantially non-absorbent foam.

Yet another embodiment of the present invention provides such a system wherein said tip is medical grade, non-absorbent, closed cell foam.

A yet further embodiment of the present invention provides such a system wherein said tip is from a material selected from the group of materials consisting of medical grade closed cell polyethylene, polyethylene/ethylene vinyl acetate co-polymers, and combinations thereof.

A further embodiment of the present invention provides such a system wherein said tip is configured with a shape selected from the group of shapes consisting of cones, frustrocones, rectangular prisms, cubes, spheroids, cylinders, and chisel tips.

An even further embodiment of the present invention provides such a system wherein said tip is of medical grade vinyl.

A yet further embodiment provides a taper adjacent to the proximal end of the dissecting tip, providing a mechanism for the deflection of mechanical forces on the dissecting tip with removal of the soft tissue dissector through a laparoscopic port.

A still further embodiment of the present invention provides a taper adjacent to the proximal end of the dissecting tip, preventing insertion of the soft tissue dissector through an inappropriately sized laparoscopic port.

An even further embodiment of the present invention provides such a system wherein said handle is configured from a material selected from metal, plastic, fiber glass, composites, and combinations thereof.

In another embodiment, the shape of the distal dissector could be altered by movement of an outer metal sheath or by internal changes due to deformation by an inner cell.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is an elevation view illustrating a soft tissue dissector configured in accordance with one embodiment of the present invention.

FIG. 2 is a perspective view illustrating a soft tissue dissector taper configured according to one embodiment of the present invention.

FIG. 3 is a perspective view illustrating a soft tissue dissector tapered cap configured according to one embodiment of the present invention.

FIG. 4A is a graphical representation of the water absorbency of a material (which may be utilized in the formation of dissection tips) and its relationship to deterioration of dissecting function with saturation.

FIG. 4B is a graphical representation of relative water absorbencies of materials which may be utilized as kittner tips.

FIG. 5A is a table providing comparative density values for various foam products.

FIG. 5B is a table providing comparative compression values for various foam products.

FIG. 5C is a table providing durometers for various foam products.

FIG. 6 is a table providing characteristics of closed cell polyethylene and polyethylene/ethylene vinyl acetate copolymer foams that are ideal for use as surgical dissector tips.

FIG. 7A is a perspective view illustrating a cylindrical tip of a soft tissue dissector configured in accordance with one embodiment of the present invention.

FIG. 7B is a perspective view illustrating a frustoconical tip of a soft tissue dissector configured in accordance with one embodiment of the present invention.

FIG. 7C is a perspective view illustrating a rectangular prismatic tip of a soft tissue dissector configured in accordance with one embodiment of the present invention.

FIG. 7D is a perspective view illustrating a chisel shaped tip of a soft tissue dissector configured in accordance with one embodiment of the present invention.

### DETAILED DESCRIPTION

One embodiment of the present invention provides a soft tissue dissector 10 configured for use in minimally invasive surgical techniques such as laparoscopy and endoscopy. Such an embodiment utilizes a dissecting tip 12 disposed on a handle 14 configured for manipulation of the tip 12 through a surgical incision. The dissecting tip 12 is configured to be durable, and substantially non-absorbent.

It is desirable that, in such an embodiment, the dissecting tip 12 of the dissector be durable, securely mounted to the handle 14, and possess sufficient rigidity to provide resistance when applied to a bodily tissue without abrading the tissue. Such resistance allows the tip 12 to be used to separate and move tissues and organs without slipping.

The handle 14 of the dissector 10 may be a straight rod, or may be configured with a curvature to permit the user greater range of motion. In an alternate embodiment, a curvature may be utilized to increase the ergonomics of surgical manipulation of the dissector. In a further embodiment, the diameter of the straight rod with attached dissecting tip would not exceed the 10 mm channel diameter of typical laparoscopic ports employed during minimally invasive surgery. Likewise, one skilled in the art can appreciate that other values can be utilized as parameters for the combined shaft-tip diameter, as laparoscopic ports can alternatively provide 12 mm, 5 mm or 2 mm channels for instrument insertion. In an embodiment utilizing a curved handle, the curvature provided to the handle would be incorporated such that the overall diameter of the dissector shaft with tip and curvature would not exceed 10 mm, in order to allow passage of the dissector through a 10 mm laparoscopic port. In alternate embodiments, the shaft-tip-curvature diameter would not exceed 12 mm, 5 mm, or 2 mm depending upon the port size through which use of the dissector is intended. Likewise, one skilled in the art can appreciate that other handle configurations may be employed, for example handles that are configured to be flexed or bent by the user either before use or by external manipulation while inserted. The handle 14 may be configured from metal, plastic, fiber glass, composites, or other suitable biocompatible material.

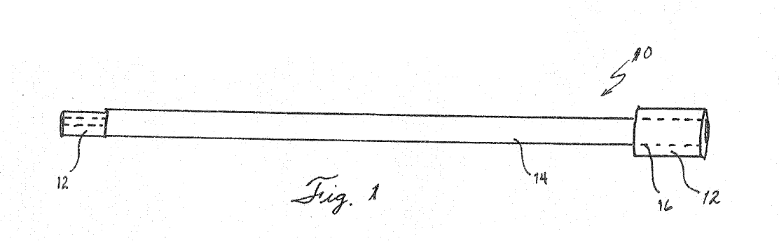

As illustrated in FIG. 1, in embodiments where no other attachments connecting to the proximal end of the handle are required, tips 12 of different sizes or shapes may be disposed at either end of the handle 14 to allow the user to readily switch to a tip more suited to a particular task. Tips of various sizes may be selected prior to surgery depending on the expected tissues and situations, or interchangeable tips may be used.

As illustrated in FIG. 1, a soft tissue dissector 10 may be configured with an elongate handle 14, and a dissecting tip 12. The dissecting tip 12 may in one embodiment be affixed to the handle using a biocompatible epoxy 16. Likewise, other means of fixation, can be employed. In an alternate embodiment, the dissecting tip can be secured to a connector that attaches to a receiving connector secured to the handle, allowing for the interchange of tips during surgery.

As illustrated in FIG. 2, in one embodiment of the present invention, a taper 20 from the dissecting tip 12 to the handle 14 is provided at the proximal end of the dissecting tip 12. This taper 20 functions to deflect mechanical forces that may be exerted on the dissecting tip 12 during removal of the soft tissue dissector 10 from a laparoscopic port or other similar surgical maneuvers. If the dissector is not properly centered within the laparoscopic port channel during removal, the edge or wall of the port can exert excessive mechanical force on the dissecting tip; said taper acts to deflect such excessive forces away from the dissecting tip. In addition, the taper 20 functions to prevent the insertion of the soft tissue dissector through an inappropriately small laparoscopic port. In one embodiment of the present invention, a dissecting tip 12 fabricated from foam would have a compressibility that could allow the insertion of the soft tissue dissector into an inappropriately small port channel. An appropriately sized non-compressible taper 20 at the proximal end of the dissecting tip would prevent the insertion of the soft tissue dissector into an inappropriately small port channel. In one embodiment of the present invention, the taper 20 would have a diameter of just less than 10 mm, for use of the soft tissue dissector through 10 mm laparoscopic ports. In other embodiments, the taper 20 would have a diameter of just less than 12 mm, 5 mm or 2 mm for use through respectively sized laparoscopic ports.

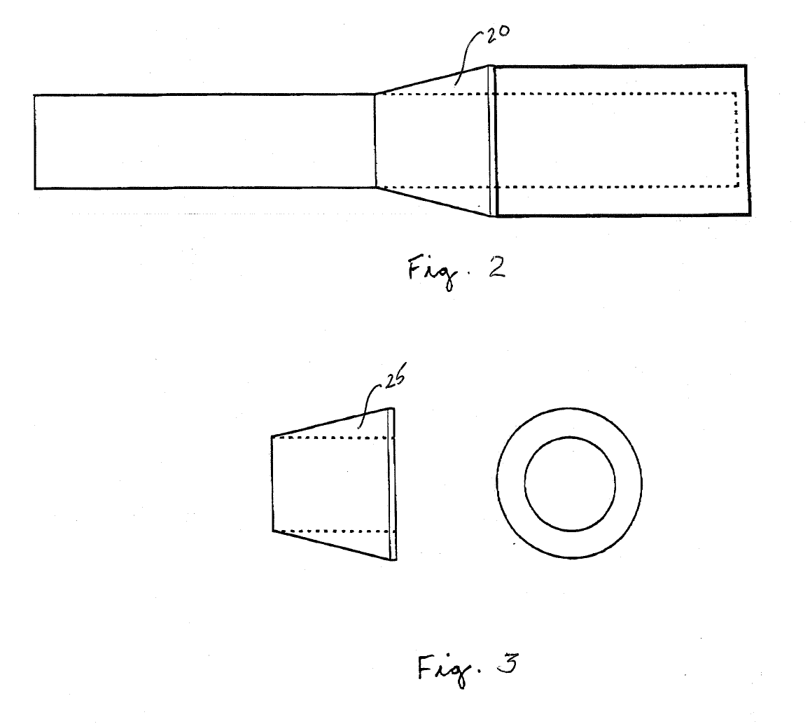

The taper 20, of one embodiment of the present invention can be constructed from a biocompatible epoxy affixed to the handle 14 just adjacent to the proximal end of the dissecting tip 12. In an alternate embodiment, the taper 20 may be affixed to both the handle 14 and the proximal end of the dissecting tip 12. In a still further embodiment, as illustrated in FIG. 3, the taper can be achieved using a distinct component 25 affixed to the handle just proximal to the dissecting tip (not shown). This component could be constructed from a rigid biocompatible material such as but not limited to stainless steel or plastic. Such a tapering component 25 would serve as a protective cap to the dissecting tip (not shown).

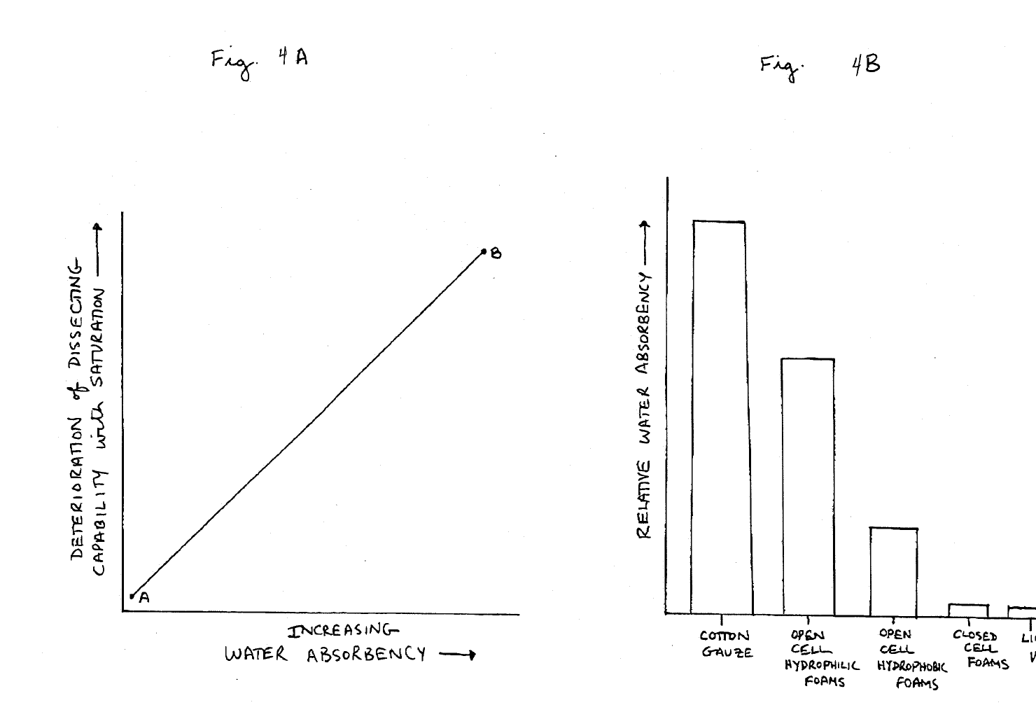

The material used in the fabrication of the dissecting tip 12 may be a medical grade, substantially non-absorbent material. The high absorbency of traditional materials used in the fabrication of kittners (ex. cotton gauze, spun cotton) results in saturation of these dissecting tips and a subsequent loss of rigidity in their structure. With loss of rigid structure, a kittner is unable to provide adequate resistance for the dissection of tissue. FIG. 4A is a theoretical graphical representation of the relationship between the water absorbency of a material and the deterioration of dissecting function seen with saturation of the material. Point A is intended to represent a minimally absorbent material, which tends to maintain its dissecting capability when exposed to a fluid environment. Point B is intended to represent a highly absorbent material, which tends to lose its dissecting capability when exposed to a fluid environment. While absorbency is a critical feature of soft tissue dissectors used in open surgery (during which oozing of blood from the microvasculature tends to obscure the surgical field), many laparoscopic procedures are performed under pneumoperitoneum. The pressure induced by the pneumoperitoneum, in addition to other clinical factors unique to laparoscopic surgery, largely precludes microvascular oozing and bleeding, thereby decreasing the need to clear the field of blood during laparoscopic procedures. Therefore, increased absorbency of the dissecting tip, which results in saturation and deterioration of functionality, may be less desirable in laparoscopic surgery as a deteriorated dissector will need to be replaced, necessitating increased exchange of instruments through laparoscopic ports. In some instances, structural deterioration of the dissecting tip may lead to a detachment and thus loss of the dissecting tip, increasing patient morbidity. Thus, materials with low water absorbency are better suited as substrates for use as dissecting tips in laparoscopic procedures, providing greater durability during these surgeries.

FIG. 4B is a graphical representation of the relative water absorbency of materials that are typically used for, or may be considered for use in the fabrication of a tip for a soft tissue dissector. The need for absorbency is greatly decreased in laparoscopic procedures; thus, materials that are substantially less absorbent than cotton gauze, which is traditionally used in the fabrication of dissecting tips, may be considered and in some instances may be better suited in the fabrication of tips for laparoscopic soft tissue dissectors according to various embodiments of the present invention. FIG. 4B illustrates the relative absorbency of some synthetic, medical grade, non-cotton based materials that could be considered in the fabrication of tips for soft tissue dissection. Some open cell polyurethane foams can be less absorbent than cotton/gauze. Hydrophilic open cell foams are manufactured with water, and thus exhibit greater absorbency than hydrophobic open cell foams, which are produced in the absence of water. Closed cell foams, such as those selected for some embodiments of the present invention are non-absorbent by nature, with only minimal absorbency seen along cut edges of these materials. The selection of such non-absorbent foams facilitates improved performance over known dissectors. Closed cell foams with smaller cell sizes display less absorbency at these cut edges. Liquid vinyls such as plastisol are also non-absorbent materials which may be used in various embodiments of the present invention. In one embodiment of the present invention, a foam with substantially non-absorbent properties is employed. In such an embodiment, the foam used displayed less than or equal to approximately 2% increase in its volume in water when placed in a water bath until saturation.

In addition to low water absorbency, other characteristics of a material configured according to one embodiment of the present invention for use as a laparoscopic soft tissue dissecting tip include adequate rigidity to separate and move tissues and organs, without causing trauma to these structures, and adequate durability. Foam based materials can provide the optimal combination of these characteristics, and in one embodiment of the present invention, the dissecting tip would comprise a foam material. Foam materials can be formulated to provide a wide range of firmness or softness and durability—to identify a foam material that would be optimal for soft tissue dissection, one must consider foam density, compressability, durometer, and other surface characteristics.

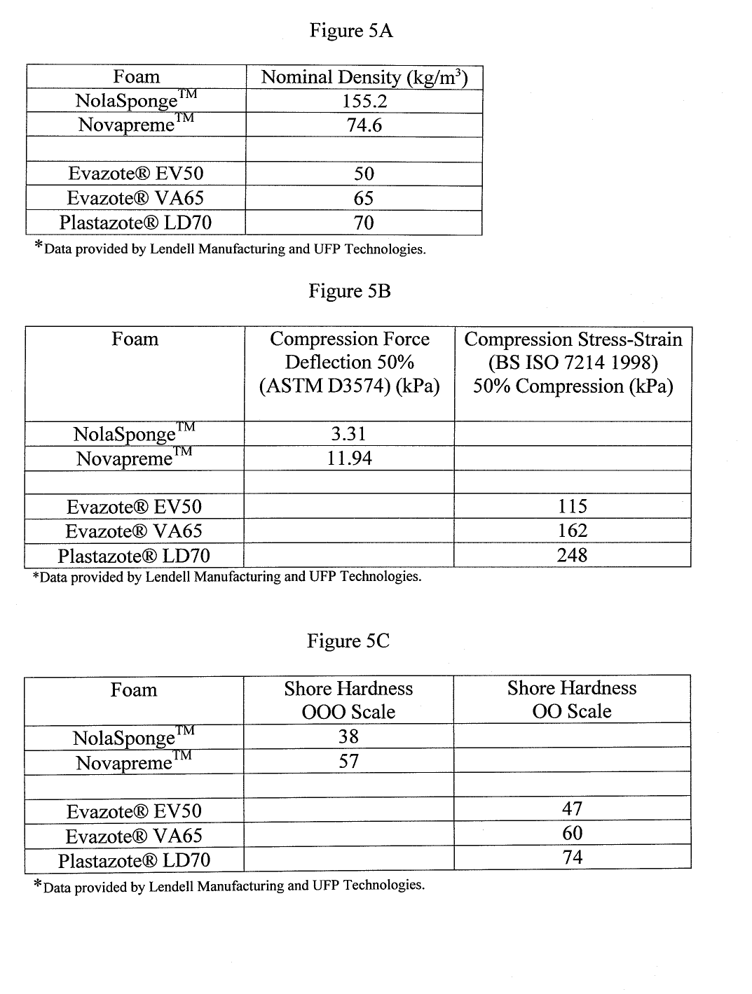

While it is commonly believed that density is a measure of foam firmness, stiffness or load bearing capacity, this is not the case—foam firmness is independent of foam density. Foam density is an indicator of the durability of a foam; higher densities indicate greater durability. FIG. 5A details the density of some foams that may be considered in the construction of a laparoscopic soft tissue dissecting tip. While all these foams possess a relatively high density, only some of these have the rigidity required for adequate soft tissue dissection.

Foam firmness or stiffness is evaluated utilizing tests that measure the compressibility of a foam, such as compression force deflection and compression stress-strain. The greater the force needed to cause compression of a foam to a given % of its original thickness, the greater the rigidity or stiffness of that foam. A soft tissue dissector needs to possess adequate rigidity both at the handle and at the dissecting tip such that when mechanical force needs to be exerted against tissues or organs for dissection, the force applied by the operator is translated to dissection of these tissues rather than to compression of the dissecting tip or deformation of the handle. Conversely, foams with too much rigidity could cause trauma to tissues and organs; thus, an appropriate level of firmness needs to be identified for optimal soft tissue dissection. FIG. 5B details compression parameters of various foams that may be considered in the fabrication of a laparoscopic dissecting tip. The Novapreme™ (methylene diphenylene diisocyanate based flexible polyurethane foam) and Nolasponge™ (toluene diisocyanate based flexible polyurethane foam) formulations provided the least rigidity for soft tissue dissection; these foams would be suited for delicate dissections that could be achieved with relatively low mechanical forces. In contrast, low density polyethylene and polyethylene/ethylene vinyl acetate copolymer foams such as the Evazote® and Plastazote® formulations were determined to have adequate firmness for soft tissue dissection over a wide range of clinical applications, with Evazote® VA65 providing optimal firmness, Evazote® EV50 providing slightly less rigidity, and Plastazote® LD70 providing a slightly higher degree of firmness.

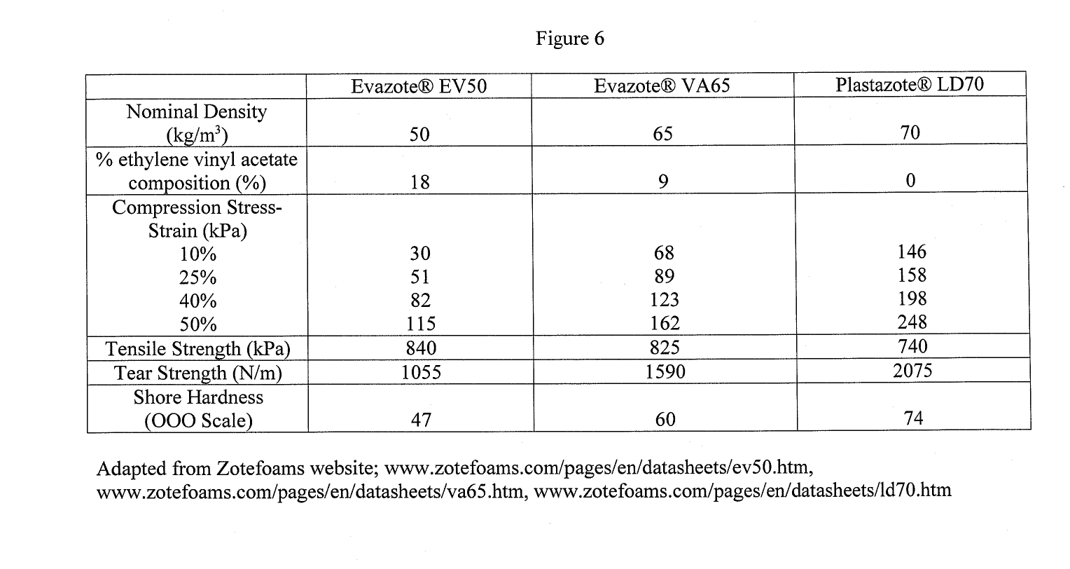

Another characteristic that must be considered in identifying an optimal foam for soft tissue dissection is durometer, or hardness of the foam. Durometer is most readily thought of as how hard or soft a foam feels. This characteristic of foam is important not only in assuring that a particular foam will not traumatize tissues, but also in identifying a foam with adequate frictional characteristics, such that the dissecting tip will not slip off of tissues during dissection. Foams that are very soft or smooth in feel tend to provide less friction during dissection; durometer is one component of assessing the surface characteristics that a particular foam would provide in this regard. FIG. 5C details durometers of various foams that may be considered in the fabrication of a laparoscopic dissecting tip. The Novapreme™ and Nolasponge™ formulations are very soft foams and are measured on the OOO Shore Hardness scale, which provides a hardness measure for the softest of foam materials. The Novapreme™ formulation not only has a low durometer, but also is composed of a polymer formulation that provides it with a smooth feel; because of these characteristics, it provides a less than optimal frictional surface for most soft tissue dissection. The Nolasponge™ formulation also has a very low durometer, but possesses reticulations that increase frictional forces at its surface. This may provide the Nolasponge™ formulation with adequate surface properties in the delicate dissection of soft tissues. The low density polyethylene and polyethylene/ethylene vinyl acetate copolymer foam formulations Evazote® and Plastazote® possess greater durometer, and thus were measured on the Shore OO Hardness scale. Evazote® EV50 has the lowest durometer of these three formulations; along with its relatively high content of ethylene vinyl acetate (FIG. 6), it possesses low frictional characteristics at its surface. However, in select applications, this formulation may be adequate for soft tissue dissection. Evazote® VA65 and Plastazote® LD 70 both possess optimal frictional characteristics at their surface for soft tissue dissection. The higher durometer and absence of ethylene vinyl acetate content in Plastazote® LD 70 provide it with a slightly coarser feel; again, the polyethylene/ethylene vinyl acetate copolymer Evazote® VA65 displayed optimal characteristics for use as a soft tissue dissecting tip.

Thus, in one embodiment of the present invention, a medical grade closed cell polyethylene/ethylene vinyl acetate copolymer material is used in the construction of the dissecting tip. Similarly, polyethylene copolymers may be used without ethylene vinyl acetate. FIG. 6 details some of the characteristics of three such foams that make them particularly well suited for use in laparoscopic dissecting tips. As discussed, the foam is selected to have a rigidity that allow it to resist excessive flexing without rupturing delicate tissues.

In a further embodiment of the present invention, a medical grade vinyl is used in the fabrication of the dissecting tip 12. Vinyl materials provide the benefits of biocompatibility, adequate rigidity and durability when used in the fabrication of dissecting tips. In one embodiment, the liquid vinyl plastisol is dip molded to produce a soft tissue dissecting tip. Dissecting tips fabricated from liquid vinyls can additionally undergo secondary operations to incorporate dimpling, striations or other surface markings at the tip surface, in order to increase the frictional surface characteristics of such materials as needed.

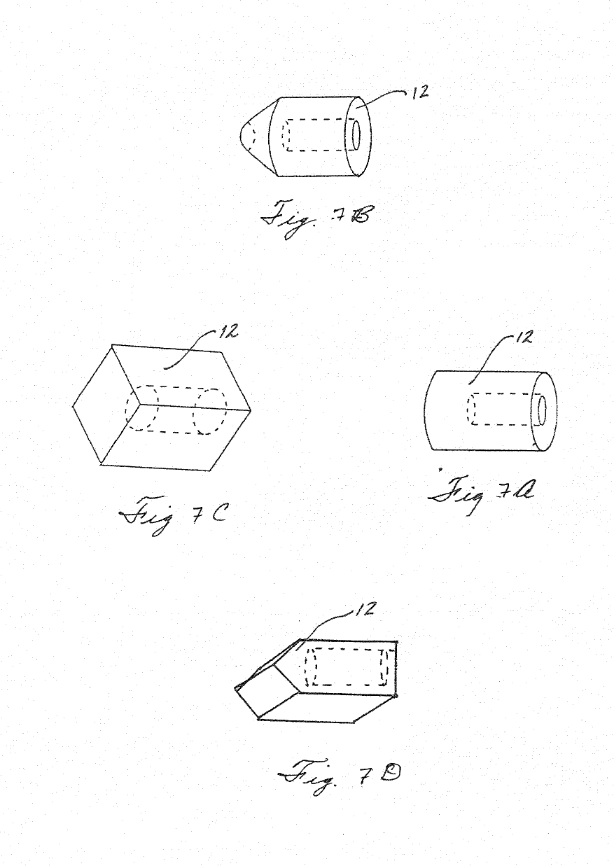

As illustrated in FIGS. 7A-D, a variety of shapes may be employed in the design of the configuration of the tip 12 of the dissector. FIG. 7A illustrates a cylinder, hollow at one end to receive the handle (Not shown). In FIG. 7B, a cylinder is provided, capped at a distal end with a frusto-conical prism. FIG. 7C illustrates a rectangular prism with its major axis coaxial with the handle (Not shown). FIG. 7D illustrates a dissector tip provided with a chisel shaped distal end. One skilled in the art will readily appreciate that other shapes may be used, such as a traditional spheroid tip, rounded tip, a conic tip, a pyramidal tip, or other shapes suited to provide the surgeon with a fine degree of dexterity required to fit the dissector between tissue planes without damaging tissues unintentionally. One skilled in the art will readily appreciate that tip shape may allow the user to reach areas previously obstructed or difficult to reach or may provide focused or diffused pressure.

One embodiment of the present invention provides a system for delicate blunt tissue dissection, the system having a elongate handle; and a synthetic, non-absorbent, first dissector tip disposed on the handle.

Another embodiment of the present invention provides such a tip made from of a substantially non-absorbent foam.

In such an embodiment the tip may be configured with a shape selected from the group of shapes consisting of cones, frustrocones, rectangular prisms, cubes, spheroids, cylinders, and chisel tips.

Such a tip may be medical grade, closed cell foam or made from a material selected from the group of materials consisting of polyethylene, polyethylene/ethylene vinyl acetate co-polymers, methylene diphenylene diisocyanate based flexible polyurethanes, toluene diisocyanate based flexible polyurethanes, plastisol, and combinations thereof. It may be sufficiently durable to resist deterioration throughout a surgical procedure.

In such a system the handle may be configured from a material selected from metal, plastic, fiber glass, composites, and combinations thereof. A second tip may, in some embodiments, be disposed at an end of the handle distal to the first tip.

The tip of the system configured according to one embodiment of the claims has absorbency not greater than 2% increase in volume at saturation.

A taper may also be disposed between the first tip and the handle. Such a taper may be configured from a rigid material and may also be a tapered collar disposed upon the handle proximal to the first tip.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

We claim:

1. A system for delicate blunt tissue dissection, the system comprising:
An elongate handle; and
A synthetic, non-absorbent, first dissector tip disposed on said handle.

2. The system of claim 1 wherein said tip is of a substantially non-absorbent foam.

3. The system of claim 1 wherein said tip is configured with a shape selected from the group of shapes consisting of cones, frustrocones, rectangular prisms, cubes, spheroids, cylinders, and chisel tips.

4. The system of claim 1 wherein said tip is medical grade, closed cell foam.

5. The system of claim 1 wherein said tip is from a material selected from the group of materials consisting of polyethylene, ethylene vinyl acetate co-polymers, methylene diphenylene diisocyanate based flexible polyurethane foam, toluene diisocyanate based flexible polyurethane foam, plastisol, and combinations thereof,.

6. The system of claim 1 wherein said tip is sufficiently durable to resist deterioration throughout a surgical procedure.

7. The system of claim 1 wherein said handle is configured from a material selected from metal, plastic, fiber glass, composites, and combinations thereof.

8. The system according to claim 1 further comprising a second tip disposed at an end of said handle distal to said first tip.

9. The system according to claim 1 wherein said tip has an absorbency not greater than 2% change in volume in water.

10. The system according to claim 1 further comprising a taper disposed between said first tip and said handle.

11. The system according to claim 10 wherein said taper is of a rigid material.

12. The system according to claim 10 wherein said taper is a tapered collar disposed upon the handle proximal to said first tip.
